(19) United States
(12) Patent Application Publication
(10) Pub. No.: US 2016/0101255 A1
(43) Pub. Date: Apr. 14, 2016

(54) Novel airway tube stabilizer

(71) Applicant: David Kumpel, Dyer, IN (US)

(72) Inventor: David Kumpel, Dyer, IN (US)

(21) Appl. No.: 14/882,184

(22) Filed: Oct. 13, 2015

Related U.S. Application Data
(60) Provisional application No. 62/063,514, filed on Oct. 14, 2014.

(51) Int. Cl. A61M 16/04

(52) U.S. Cl. CPC A61M 16/0497; A61M 16/049; A61M 16/0434; A61M 2205/0216; A61M 2205/273

(57) ABSTRACT

A novel tube stabilizer with a collar 1 and an aperture 2. A tube 225 from a medical airway device 210, can pass through the aperture 2. The stabilizer slides along the tube 225 and fits into the mouth of a patient on which an airway device 210 is being use during a medical procedure.

### CROSS-REFERENCE TO RELATED APPLICATIONS

The present application claims the benefit of U.S. Provisional Patent Application No. 62/063,514, filed Oct. 14, 2014, the entirety of which is hereby incorporated by reference.

### BACKGROUND

An airway device, such as a supra-glottic airway, is used by medical professionals and anesthesia providers for establishing breathing paths, airways, in unconscious patients. They are also used for administering and monitoring anesthesia in unconscious patients. Airway devices can also be used in emergency situations when ventilation is necessary. Generally, an airway device comprises a sealing portion and an airway tube which work together to establish an airway in a patient, intubation. They are used for one goal, to establish an open airway in a patient.

These airway devices have worked well over the years but problems still occur during their use. To keep a reliably working airway in a patient, a good seal around the laryngeal inlet or the glottis at all times during use of the device is crucial. This means that the airway device must stay in position, remaining stable, throughout the patient's surgery or other medical procedure. This stability becomes difficult to maintain when the patient involuntarily moves while anesthetized. Such movements cause the airway device to become dislodged or to shift positions which causes a breaking of the path for air. Use of an intubation tube on patients having few or no teeth poses additional stability problems. Loss of a patient's airway is a life threatening situation.

Additionally, the loss of stability in an airway device causes distractions during the medical procedure because of the need for airway device adjustments to get the patient breathing again. Such distractions are time-consuming, difficult for medical staff and cause great danger to the patient. There is therefore a need for a device that can keep an airway device stably in place during medical procedures which involve the use of an airway device to keep a patient's airway clear.

It is well known that a poorly fitted supra-glottic airway is not only unsafe, but can become a medical emergency if oxygen cannot be delivered safely to a patient. In fact, the number 1 cause of anesthesia related deaths is airway emergencies becoming airway disasters resulting in patient deaths.

Information relevant to attempts to address these problems can be found in U.S. Pat. No. 7,134,431 B2. However, this reference suffers from one or more of the following disadvantages: the tab method does not provide enough surface area to benefit from the patient's body support and the tab does not work well on edentulous patients. Also, tape around the mouth and chin is also used to provide stabilization of the tube but tape can lose its adhesiveness and the tube still fits poorly and requires adjusting, which can cause dislodging.

There is a need in the industry for an apparatus that is easily used with an airway device, takes advantage of the patient's body support, and stabilizes the airway device. The need is accentuated with respect to patients that do not have teeth and there is an additional necessity to fill the void left by missing teeth that might otherwise assist in stabilizing an airway device.

### SUMMARY

The present invention is directed to an apparatus that satisfies this need. I have invented a novel airway tube stabilizer comprising a collar and an aperture. A preferred embodiment has its collar being made from a soft medical grade thermoplastic elastomer (TPE). The collar fits and finds support from a patient's mouth area. It fits snugly around a laryngeal mask and is placed just under the upper and lower lip of a patient. A tube from an airway device, such as that used on human patients in medical procedures or on animals in veterinary procedures, for example a laryngeal tube, or the like, easily passes into and through the aperture allowing for easy positioning of the apparatus. The apparatus is especially useful for providing stability to airway devices when used on edentulous patients however it can be used anytime an airway device with a tube inserted into the patient's throat is used. The stability provided by the apparatus can be achieved through its shape, to name one example of how the apparatus provides stability to a tube.

Another embodiment of the present invention can be used as a stabilizer for an airway tube used on a medical patient. The tube is preferred to be a laryngeal tube but certainly can work on any other tube that is inserted into the mouth of a patient that seals within against the patient's body to provide an unobstructed path, for example a laryngeal tube providing a clear path for oxygen to pass into a patient's trachea. The embodiment includes a collar segment, it somewhat resembles a mouthpiece that is used in sporting activities. The collar segment has at least one first opening that is generally in the center of the collar segment because that is where the collar segment material provides the most lateral support to the walls of the first opening, or aperture. The at least one first opening can receive the airway tube, the airway tube can pass through the opening. When used the collar segment is removably attached to an airway tube. Preferably this is done by passing the tube through the first opening and the collar segment sliding along the tube. However, the collar segment could also have a snap or hinged design that could allow the collar segment to be placed onto the tube without having to remove the oxygen circuit. The collar segment engages the patient's lips by being placed generally under the upper and lower lips. This is especially useful when the patient has no teeth. Because patients will vomit or expel other material while in surgery or need to have other medical apparatus' inserted into the oral cavity, the collar segment has at least one second opening. The path of for oxygen that the tube takes generally traverses the patient's oral cavity, then goes through the patient's larynx, and then up to the patient's trachea. Oxygen can then make its way into the patient's trachea.

The collar segment prevents the airway tube from moving and breaking the clear path for oxygen to pass. This is because there is stability in the design of the collar segment that secures the tube and prevents it from becoming dislodged or needing readjustments. The stabilizer is best made for only a single use and manufactured from a TPE material.

Another embodiment of the present invention works as an intubation tube positioning device that is used on medical patients, human or animal. The device includes a collar segment that has a medial aperture. That medial aperture is configured to accept an intubation tube. The collar segment is shaped to accommodate upper and lower lips of a patient, and finds some support from the patients gums.

A novel method embodiment of my invention includes a positioning and stabilizing a laryngeal intubation tube that is part of a supra-glottic mask, many time referred to as a laryngeal mask airway. The tube is positioned centrally in a patient's mouth during intubation through the use of an intubation mouthpiece. The mouthpiece includes a medial aperture configured to accept an end of the intubation tube. This method for using it involves advising the patient to go to sleep before they undergo the procedure. Then covering the patient's mouth with an oxygen mask connected to a breathing circuit, machinery common in the anesthesia industry, that delivers oxygen. Then delivering anesthesia to the patient via an IV to put the patient to sleep. After the patient falls asleep and is no longer breathing, when apnea sets in, then the user removes the circuit and oxygen mask that is covering the patient's mouth. Using fingers to scissor the patient's mouth open, the laryngeal intubation tube that is part of a supra-glottic mask is inserted into the patient via the patient's mouth to create a seal between the supra-glottic mask and the patient's body just above the patient's glottis. Then the user attaches the circuit to the laryngeal intubation tube that is part of a supra-glottic mask and checks that the patient's chest is rising to indicate that oxygen is entering the patient's lungs, indicating a positive end tidal CO2. Next is to quickly disconnect the circuit from the laryngeal intubation tube that is part of a supra-glottic mask. Then attaching the mouthpiece to fit snugly around the laryngeal intubation tube that is part of a supra-glottic mask by passing the exposed end of the intubation tube through the medial aperture. Then the use will reconnect the circuit to the laryngeal intubation tube that is part of a supra-glottic mask. Then sliding the mouthpiece so that is sits under the patient's upper and lower lips providing sufficient support to the laryngeal intubation tube that is part of a supra-glottic mask to prevent breaking the seal. If necessary, the user can continue by taping the patient's chin and mouth to provide added support to the laryngeal intubation tube that is part of a supra-glottic mask.

Another embodiment of the present invention is an intubation system used on medical patients. It comprises a laryngeal intubation tube being inserted into a larynx with a tube end protruding from the mouth of a patient. There is an intubation tube mouthpiece configured to fit between upper and lower lips of the patient. The intubation tube mouthpiece includes a medial aperture sized to fit the intubation tube, wherein the tube end passes through the medial aperture and the mouthpiece is seated between the upper and lower lips of the patient. The intubation tube mouthpiece having at least one second aperture providing access to and from the patient's oral cavity. This system can have the mouthpiece made for only a single use made from TPE material or the like.

The material should be a soft pliable material that is comfortable to use yet is sturdy. The material should maintain its shape when manipulated and is made of such a material as to sufficiently allow the invention to meet the requirements of providing stability to airway devices used on patients in surgery as discussed herein.

One embodiment of the present invention relates to a novel tube stabilizer used, for example, as an accessory to airway devices and more particularly to a medical device having a collar disposed around an airway device's tube, or the like, for controlling the airway device's position and more specifically to such a stabilizer in which the collar with a hole that is adapted to be slidable along the tube of the airway device and sufficiently fitting the rima oris of a patient being ventilated.

The size of one embodiment of the invention can be made to vary in several respects depending on the size of the person or animal being treated.

### BRIEF DESCRIPTION OF THE SEVERAL VIEWS OF THE DRAWINGS

These and other features, aspects, and advantages of the present invention will become better understood with regard to the following description, appended claims, and accompanying drawings where:

FIG. 1 shows a perspective view of one embodiment of the invention.

FIG. 2 shows a perspective view of another embodiment of the invention being used with an intubation tube.

### DESCRIPTION

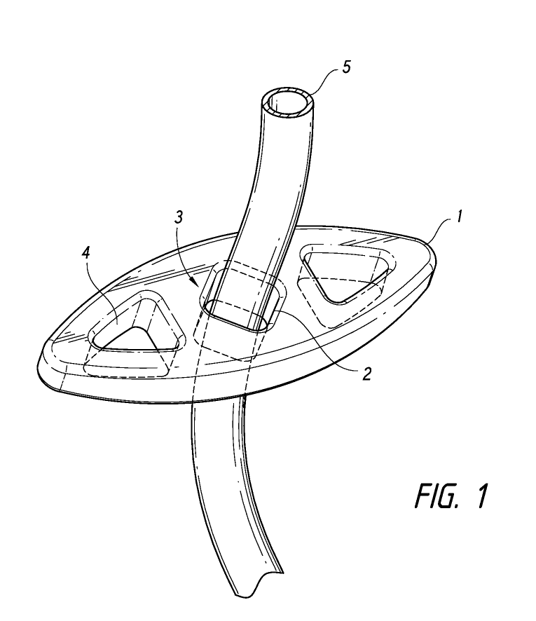

As shown in FIG. 1, a perspective view of one embodiment of the invention comprises a collar 1, the collar 1 having an aperture 2. An airway tube 5 passing through the aperture 2. The aperture 2 keeping a snug fit on the airway tube 5 but yet allowing for sliding. The collar 1 having at least one hole 4. The aperture 2 is shaped 3 to accommodate differently sized airway tubes 5.

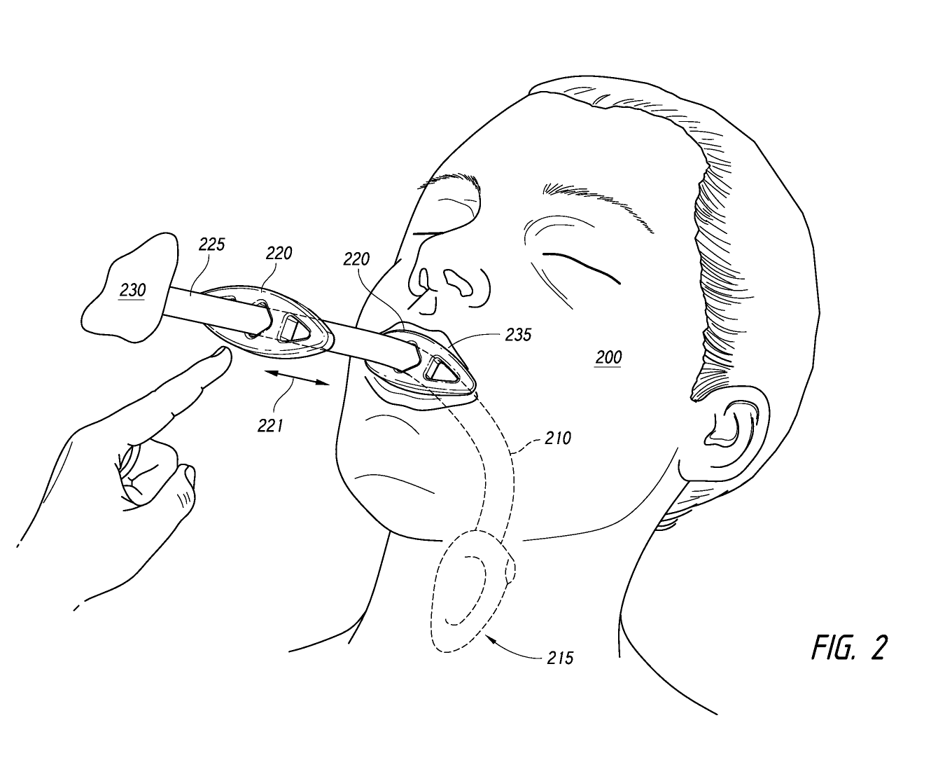

As shown in FIG. 2, a perspective view of another embodiment of the invention being used with an airway device comprises a patient 200 that has been fitted with an airway device 210. The airway device 210 seals around the patient's laryngeal opening or glottis 215. The airway device having a tube 225 that travels outside of the patient to ventilation equipment 230. The collar 220 being moveable 221 along the tube 225. Generally a medical provider will slide the collar 221 into position. In one position the collar 220 is fit into and around the patient's mouth 235 under the patient's 200 lips filling the void and stabilizing the airway device 210.

Although the present invention has been described in considerable detail with the reference to certain preferred versions thereof, other versions are possible. For example, the collar of the invention could be hinged or semi-closed and allow for opening to fit around a tube or being pressed to snap onto the tube instead of being slid over the tube. Therefore, the spirit and scope of the appended claims should not be limited to the description of the preferred versions contained herein.

Any element in a claim that does not explicitly state “means for” performing a specified function, or “step for” performing a specific function, is not to be interpreted as a “means” or “step” clause as specified in 35 U.S.C. §112, ¶6. In particular, the use of “step of” in the claims herein is not intended to invoke the provisions of 35 U.S.C. §112, ¶6.

What I claim is:

1. A stabilizer for an airway tube used on a medical patient, the stabilizer comprising:
a collar segment;
the collar segment being made of a pliable material;
the collar segment having at least one first opening;
the at least one first opening capable of receiving the airway tube;
the collar segment being removably attached to the airway tube;
the collar segment engaging the patient's lips;
the collar segment having at least one second opening;
the airway tube being one that is commonly used in medical procedures when the patient requires a clear path for oxygen to pass, the path generally traversing the patient's oral cavity, then through the patient's larynx, and then into the patient's trachea;
the collar segment preventing movement of the airway tube which would cause breaking of the clear path for oxygen to pass.

2. The stabilizer of claim 1 wherein the stabilizer is made for only a single use.

3. The stabilizer of claim 1 wherein the pliable material is TPE.

4. An intubation tube positioning device used on medical patients, the device comprising:
a collar segment;
the collar segment having a medial aperture;
the medial aperture configured to accept an intubation tube;
the collar segment being shaped to accommodate a space under the upper and lower lips of a patient.

5. A method of positioning and stabilizing a laryngeal intubation tube that is part of a supra-glottic mask centrally in a medical patient's mouth during intubation utilizing an intubation mouthpiece, the mouthpiece including a medial aperture configured to accept an end of the laryngeal tube, the method comprising:
preparing a medical patient to be put to sleep before a medical procedure that requires the use of anesthesia;
covering the patient's mouth with an oxygen mask connected to a breathing circuit that delivers oxygen;
delivering anesthesia to the patient via an IV to put the patient to sleep;
after the patient falls asleep and is no longer breathing then removing the breathing circuit and the oxygen mask that is covering the patient's mouth;
using fingers to open the patient's mouth, the laryngeal intubation tube that is part of a supra-glottic mask is inserted into the patient via the patient's mouth to create a seal between the supra-glottic mask and the patient's body just above the patient's glottis;
attaching the oxygen circuit to the laryngeal intubation tube that is part of a supra-glottic mask and checking that the patient's chest is rising to indicate that oxygen is entering the patient's lungs, indicating a positive end tidal CO2;
quickly disconnecting the oxygen circuit from the laryngeal intubation tube that is part of a supra-glottic mask;
attaching the intubation mouthpiece to fit snugly around the laryngeal intubation tube that is part of a supra-glottic mask by passing the exposed end of the intubation tube through the medial aperture;
reconnecting the circuit to the laryngeal intubation tube that is part of a supra-glottic mask;
sliding the mouthpiece so that it sits under the patient's upper and lower lips providing sufficient support to the laryngeal intubation tube that is part of a supra-glottic mask to prevent breaking the seal;
taping the patient's chin and mouth to provide additional support to the laryngeal intubation tube that is part of a supra-glottic mask.

6. An intubation system used on medical patients, the system comprising:
a laryngeal intubation tube;
the intubation tube being inserted into a larynx with a tube end protruding from the mouth;
an intubation tube mouthpiece configured to fit between upper and lower lips of the patient;
the intubation tube mouthpiece including a medial aperture sized to fit the intubation tube, wherein the tube end passes through the medial aperture and the mouthpiece is seated under the upper and lower lips of the patient;
the intubation tube mouthpiece having at least one second aperture;
the at least one second aperture providing access in and out of the patient's oral cavity.

7. The stabilizer of claim 6 wherein the mouthpiece is made for only a single use.

8. The stabilizer of claim 6 wherein the mouthpiece is made from TPE material.
